(19) United States
(12) Patent Application Publication
(10) Pub. No.: US 2026/0290503 A1
(43) Pub. Date: Sep. 24, 2026

(54) MEDIUM-DEPTH WHOLE GENOME BISULFITE SEQUENCING ANALYSIS SYSTEM THAT INTEGRATES CLASSIFICATION MODELS TRAINED ON DISPARATE DATASETS

(71) Applicant: OMICXHEALTH, INC., San Ramon, CA (US)

(72) Inventor: Mythili SUBHARAM, San Ramon, CA (US)

(73) Assignee: OMICXHEALTH, INC., San Ramon, CA (US)

(21) Appl. No.: 19/572,180

(22) Filed: Mar. 19, 2026

Related U.S. Application Data
(60) Provisional application No. 63/775,222, filed on Mar. 20, 2025.

(51) Int. Cl. G16B 30/00; G16B 40/20; G16B 40/30; G16H 50/20

(52) U.S. Cl. CPC G16B 30/00; G16B 40/20; G16B 40/30; G16H 50/20

(57) ABSTRACT

A system that analyzes medium-depth WGBS data from a patient to test for any of a potentially large number of health conditions. The system leverages existing disparate genomic datasets to train a set of probabilistic classifiers; this allows for rapid deployment of many models without collecting a huge training set of WGBS data. Patient WGBS data is then transformed into values that are compatible with these models. Data processing may begin by selecting models based on patient health status, history, demographics, and clinician input. Raw data is aggregated into region-level methylation percentages for regions corresponding to the selected models, allowing the system to use medium-depth or low-depth WGBS data, thereby significantly reducing the expense of sequencing. Probability outputs from selected models may be combined using a rules engine that generates overall clinical conclusions. Snapshots generated from WGBS data enable retrospective auditing and iterative training of novel classification models.

This application claims priority to U.S. Provisional Patent Application Ser. No. 63/775,222, filed 20 Mar. 2025, the specification of which is hereby incorporated herein by reference.

### BACKGROUND OF THE INVENTION

### Field of the Invention

One or more embodiments of the invention are related to the field of genomic data analysis. More particularly, but not by way of limitation, one or more embodiments of the invention enable a medium-depth whole genome bisulfite sequencing (WGBS) analysis system that integrates classification models trained on disparate datasets. One or more embodiments of the invention may extract epigenetic methylation features from aligned whole genome sequencing data, and may integrate an ensemble of complementary specialized classification models trained on disparate datasets and adjudicated by a hierarchical rules engine to resolve oncogenic signals. One or more embodiments may also incorporate structural fragmentomics features derived from WGBS data to confirm or update clinical conclusions.

### Description of the Related Art

Conventional liquid biopsy diagnostics typically utilize a single global classifier or a single model per omic layer. A significant technical limitation of such single-model architectures is the requirement to optimize for a single, global decision boundary across a diverse patient population. This creates an inherent technical conflict: increasing sensitivity for early-stage (for example, Stage 1 cancer) detection inevitably captures non-malignant biological noise (e.g., cirrhosis or inflammation), leading to a failure in clinical specificity. These architectures may suffer from ‘feature interference,’ where rare, high-variance markers for early malignancy are mathematically masked by the dominant signatures of the broader training population.

Furthermore, while targeted panel assays are developed using whole genome discovery cohorts, the resulting clinical tests are physically restricted to a static set of genomic probes. This ‘frozen’ marker set lacks visibility into the broader genome during prospective clinical use, preventing the system from resolving unanticipated biological variations or etiology-specific noise that may arise in large-scale, real-world populations. Conversely, traditional whole genome bisulfite sequencing (WGBS) has historically faced economic barriers due to the high sequencing depth required for single-site resolution. There is a technical need for a system that leverages the comprehensive breadth of the whole genome in a clinical production environment while achieving high accuracy at medium sequencing depths (e.g., 10x-30x) through integration of classification models trained on disparate datasets.

The following examples of existing diagnostic systems illustrate some of the limitations of these systems, and identify how one or more embodiments of the invention may address these limitations:

(1) Multi-Modal Ensembles with Flat Methylation Feature Sets. Current diagnostic systems utilize multi-modal ensembles to integrate epigenetic and structural signals for single-disease detection. These platforms typically employ a flat methylation feature set, wherein a global model processes a unified vector of methylation data to identify a specific condition.

Technical Limitation: Flat models may lack the granularity to resolve biological mimicry (e.g., cirrhosis-induced noise). Because they apply a global calculation to a single, unified feature set, they cannot effectively decouple target signatures from systemic biological interference.

In one or more embodiments of the invention, the system may replace the flat model with a Mixture-of-Experts (MoE) framework comprising multiple partitioned methylation specialists. By dividing known differentially methylated regions (DMRs) into specialized, non-overlapping feature sets, the system may execute parallel experts optimized for distinct biological contexts. This may be coordinated by a Hierarchical Rules Engine that optionally incorporates a structural fragmentomics specialist to enable deterministic adjudication and resolve biological noise.

(2) DMR Discovery and Statistical Segmentation Engines. Some systems in the prior art (e.g., focus on the identification and discovery of differentially methylated regions (DMRs) from raw sequencing data. (Examples include BiSeq, described in Hebestreit, K., Dugas, M., and Klein, H. U., “Detection of significantly differentially methylated regions in targeted bisulfite sequencing data,” Bioinformatics, Vol. 29, No. 13, pp. 1647-1653 (Jul. 1, 2013); and MethylScore, described in Hüther, P., Hagmann, J., Nunn, A., et al., “MethylScore, a pipeline for accurate and context-aware identification of differentially methylated regions from population-scale plant whole-genome bisulfite sequencing data,” Quantitative Plant Biology, Vol. 3, e19, pp. 1-13 (Sep. 21, 2022).) These systems utilize unsupervised machine learning or statistical segmentation to produce a list of genomic coordinates where methylation states differ between cohorts.

Technical Limitation: These tools are primarily “Discovery Engines” that stop at the identification of relevant regions. They do not provide an infrastructure for the operational orchestration of these regions. They lack the logic to group known regions into specialized models or to dynamically route data to etiology-specific experts.

Unlike these discovery-centric engines, one or more embodiments of the invention may utilize pre-defined, validated DMRs but may also partition them into discrete feature sets assigned to specialized experts. This may allow the system to move beyond “finding a marker” to “adjudicating a diagnosis” through a multi-expert consensus that is specifically designed to handle the heterogeneity of real-world clinical samples.

(3) Rank-Based and Meta-Analytic Intersection Frameworks. Other frameworks identify an intersection of common features across disparate datasets to ensure biomarker robustness.

Technical Limitation: These frameworks act as static filters. By focusing on the “intersection” of common signals, they often smooth over the etiology-specific variations required for high-specificity diagnostics.

Rather than seeking a common intersection, one or more embodiments of the invention may maintain a modular library of specialists. This preserves the unique, non-overlapping signals required for etiology-specific resolution, which are typically lost in “intersecting” models.

(4) Probabilistic Imputation for Data Integration. Existing frameworks designed to harmonize genomic data from disparate platforms often utilize probabilistic imputation to “fill in” missing CpG values or normalize data across coverage gaps (e.g., when integrating array data with sequencing data).

Technical Limitation: Imputation is inherently probabilistic and introduces estimation risk into clinical diagnostics. More importantly, imputation models are designed to predict missing values but lack the mechanism to resolve systemic scale-shifts (differences in the overall distribution and magnitude of methylation percentages) between disparate assays. They assume the coordinate systems of the two datasets are already aligned, which is rarely true in multi-platform clinical environments.

One or more embodiments of the invention may incorporate an active Informatics Translation Layer that may perform a deterministic, two-stage coordinate transformation. Rather than “guessing” data via imputation, the system may apply a Mean-Shift Alignment. A first offset may align validation calibration samples with disparate training sets, and a conditional second offset may resolve inter-batch drift. This enables the robust application of existing research models by actively “shifting” the patient data into the model's native coordinate system, resolving scale-shift issues that imputation cannot address.

(5) Regional Aggregation as a Statistical Bridge for Low-Depth WGBS. The use of fixed-width “Binning” or biologically defined “Methylation Control Blocks” (MCBs) is a known technique for identifying co-regulated genomic regions for biomarker discovery. These methods traditionally aggregate neighboring CpG sites to find stable biological signals in high-depth or targeted sequencing datasets (see for example Xu, R. H., et al., “Circulating cell-free DNA methylation markers for early detection of hepatocellular carcinoma,” Nature Materials, Vol. 16, No. 11, pp. 1155-1161 (November 2017).).

Technical Limitation: At lower sequencing depths (10x-30x), the stochastic nature of whole-genome shotgun sequencing leads to sampling dropout, where individual CpG sites frequently have zero or insufficient read coverage. This “read density deficit” makes it impossible to calculate a stable methylation percentage for individual sites, rendering the data incompatible with models trained on high-depth, targeted assays.

One or more embodiments of the invention may utilize Regional Aggregation specifically as a technical mechanism to resolve this read-density deficit. By pooling reads across contiguous co-methylated regions, the system ensures a sufficient read-sum to calculate a stable, deterministic methylation percentage. This non-obvious application of regional aggregation acts as a statistical bridge, allowing cost-effective medium-depth sequencing to maintain parity and functional compatibility with high-depth targeted training data that would otherwise be unusable at these depths.

For at least the limitations described above there is a need for a medium-depth whole genome bisulfite sequencing analysis system that integrates classification models trained on disparate datasets.

### BRIEF SUMMARY OF THE INVENTION

One or more embodiments described in the specification are related to a medium-depth whole genome bisulfite sequencing (WGBS) analysis system that integrates classification models trained on disparate datasets.

One or more embodiments of the invention may enable a system that utilizes regional feature aggregation to achieve data efficiency at medium sequencing depths. By integrating methylation signals across defined genomic windows rather than single-site modifications, the system may increase the signal-to-noise ratio, allowing for high-confidence detection from lower read volumes.

In one or more embodiments, the system may employ a Mixture of Experts (MoE) architecture. While MoE is a recognized principle in machine learning theory for resolving local versus global manifold conflicts, its application here, specifically to resolve biological mimicry and etiology-specific interference in genomic data, is novel. The architecture may utilize an ensemble of specialized experts for a single disease state, including etiology-specific specialists (e.g., HCC models optimized for HBV-positive vs. HBV-negative backgrounds), high-sensitivity experts tuned for early-stage detection, high specificity fragmentomics experts, and malignancy auditors.

These experts may be adjudicated via a hierarchical rules engine applying non-linear conditional logic. This may enable a ‘conservative veto’ where a quiet signal from a structural malignancy auditor suppresses a high-sensitivity expert ensuring that high sensitivity for early detection does not come at the expense of clinical specificity. At the same time, high sensitivity is maintained by allowing the high sensitivity expert to shine when the malignancy auditor is unsure. By maintaining whole-genome visibility, the system may provide a dynamic diagnostic platform capable of adjudicating complex oncogenic signals that remain invisible to static, panel-based assays.

In one or more embodiments, the system may further improve specificity by utilizing cross-disease negative training, wherein datasets from non-target health conditions are used as negative examples to train the specialized experts, thereby reducing false positives from biological mimicry. A meta-classifier may utilize patient-specific clinical data to dynamically select the most relevant ensemble of experts, while a hierarchical rules engine may adjudicate results across both intra-disease (e.g., HCC-specific) and inter-disease (e.g., abdominal disease) experts to provide a comprehensive diagnostic verdict. Following selection of experts by the meta-classifier, a mathematical union of the specific genomic coordinates required by the ensemble to perform a single-pass selective extraction may be calculated; this technical configuration may reduce computational overhead and represents an improvement in the functionality of the diagnostic computer system.

The ‘Sequence Once, Analyze Many Times’ framework enabled by one or more embodiments of the invention may result in improved operational and computational efficiency. A high-fidelity, whole-genome digital methylome generated from a single physical sample may be iteratively processed by a plurality of independent diagnostic models. The diagnostic scope of the system may be increased (e.g., adding a new cancer model) or biomarkers may be refined in software without requiring a new wet-lab assay or a change in the established sequencing protocol.

One or more embodiments of the invention may include multiple classification models, a meta-classifier, a features adapter, and a health conditions engine. Each classification model may include a health condition; input features, each of which includes a methylation percentage associated with a portion of a genome and a genome region containing this portion of the genome, where the genome region is used with whole genome sequencing data; an output that include a probability that a patient has the health condition; training sample, where each training sample has a sample patient, the input features associated with the sample patient, and a label indicating whether the sample patient has the health condition; and a machine learning model trained on the training samples to calculate the output from the input features. The meta-classifier may receive context information associated with a patient, which may include one or more of health data associated with the patient, demographic data associated with the patient, and clinician orders associated with the patient. The meta-classifier may select one or more selected models from the classification models based on the context information, and it may identify selected regions of the genome as the union of genome regions associated with all input features associated with the selected models. The features adapter may receive methylation data generated by whole genome bisulfite sequencing of one or more biological samples from the patient, aggregate this data in the selected regions to calculated a methylation percentage for each selected region, and for each selected model, translate the methylation percentage for each selected region in each selected model into a value that is compatible with the training samples associated with each selected model, to yield patient feature values for the input features associated with each selected model. The health conditions engine may input the patient feature values into the selected models to calculate probabilities that the patient has the health conditions associated with the selected models.

In one or more embodiments of the invention may have a rules engine that applies one or more rules to the probabilities that the patient has the health conditions to generate conclusions about the health status of the patient.

In one or more embodiments, the meta-classifier may select multiple models, and the rules engine may combine binary verdicts form these multiple models into a union of the binary verdicts as the conclusions about the health status of the patient.

In one or more embodiments, the machine learning model may include one or more of XGBoost, a decision tree, a random forest, logistic regression, and a neural network.

In one or more embodiments, the whole genome bisulfite sequencing may be medium-depth sequencing. For example, the sequencing depth may be between 10x and 30x, inclusive.

In one or more embodiments, when the portion of the genome associated with an input feature of a classification model is a single base address, the genome region containing the portion of the genome may be a contiguous region of base addresses that contains the single base address.

In one or more embodiments, translating the methylation percentage for each selected region into a value that is compatible with the training samples may include adjusting the methylation percentage for a difference between the whole genome bisulfite sequencing of tissue samples from the patient and whole genome bisulfite sequencing of a set of calibration samples, and further adjusting the methylation percentage for a difference between the whole genome bisulfite sequencing of the set of calibration samples and the training samples.

In one or more embodiments, adjusting the methylation percentage for a difference between the whole genome bisulfite sequencing of tissue samples from the patient and whole genome bisulfite sequencing of the set of calibration samples may include: calculating a patient average methylation percentage in one or more biologically invariant regions from the whole genome bisulfite sequencing of tissue samples from the patient, calculating a calibration average methylation percentage in these one or more biologically invariant regions from the whole genome bisulfite sequencing of the set of calibration samples, and adding the difference between the calibration average methylation percentage and the patient average methylation percentage to the methylation percentage for each selected region.

In one or more embodiments, when the difference between the calibration average methylation percentage and the patient average methylation percentage is greater than a valid range of values, the system may mark the whole genome bisulfite sequencing of one or more biological samples from the patient as invalid.

In one or more embodiments, further adjusting the methylation percentage for a difference between the whole genome bisulfite sequencing of the set of calibration samples and the training samples may include: calculating a calibration average methylation percentage in the methylation percentage for each selected region from the whole genome bisulfite sequencing of the set of calibration samples, calculating a training set average methylation percentage for each selected region from the training samples wherein the sample patient does not have the health condition, and adding the difference between the training set average methylation percentage and the calibration average methylation percentage to the methylation percentage for each selected region.

In one or more embodiments, the training samples associated with at least one classification model may include additional training samples from a different classification model, wherein a sample patient associated with the additional training samples has the health condition associated with the different classification model and is labelled as not having the health condition associated with the classification model.

In one or more embodiments, the features adapter may extract and process methylation data only in the union of genome regions associated with all input features associated with the selected models.

In one or more embodiments, the meta-classifier may also select a fragmentomics model. The fragmentomics model may calculate one or more fragmentomics feature values from the whole genome bisulfite sequencing of one or more biological samples from the patient, and the rules engine may apply one or more rules to the probabilities that patient has the health conditions and to the one or more fragmentomics feature values.

In one or more embodiments, the fragmentomics feature values may include a measure of a ratio of short fragments to long fragments in the whole genome bisulfite sequencing of one or more biological samples from the patient.

In one or more embodiments, the fragmentomics model may divide the human genome into one or more series of genome bins, and the measure of the ratio of short fragments to long fragments may include the number of bins wherein a normalized z-score of a ratio of short fragments to long fragments within the bin exceeds a threshold value.

In one or more embodiments, the normalized z-score may be calculated using a mean value and a standard deviation value within said genome bin calculated using genome data from a set of healthy patients.

In one or more embodiments, the normalized z-score may be calculated using a floor value for the standard deviation within each genome bin.

One or more embodiments of the invention may also include an archive database with the whole genome bisulfite sequencing of one or more biological samples from multiple patients, and one or more additional models trained on the archive database.

In one or more embodiments, one or more of the additional models may use one or more new features extracted from whole genome bisulfite sequencing data that represent previously unknown biomarkers for health conditions associated with the additional models.

### BRIEF DESCRIPTION OF THE DRAWINGS

The above and other aspects, features and advantages of the invention will be more apparent from the following more particular description thereof, presented in conjunction with the following drawings wherein:

FIG. 1 shows an architectural block diagram of an illustrative embodiment of the invention.

FIG. 2 identifies selected factors that contribute to the invention providing broad and accurate diagnostic power at a lower cost.

FIG. 3 illustrates aggregation of WGBS data into region-level methylation percentages, which enables embodiments of the invention to work effectively with medium-depth sequencing data.

FIG. 4 illustrates potential issues in adapting existing datasets based for example on targeted assays to WGBS data.

FIG. 5 illustrates solutions to the issues shown in FIG. 4, wherein portions of the genome are expanded to sufficiently wide regions for WGBS analysis.

FIG. 6 illustrates training of a classification model on the dataset of FIG. 4.

FIG. 7 shows a variation of classification model training that uses datasets from another health conditions as negative examples, thereby improving the specificity of trained model.

FIG. 8 illustrates an approach to adapt WGBS data to the training dataset used to train a classification model.

FIG. 9 continues the example of FIG. 8 to show the feature values from WGBS data that are input into the classification model trained on a different dataset.

FIG. 10 shows illustrative components of a meta-classifier for the invention that selects models based on patient and clinician data.

FIG. 11 illustrates addition of a fragmentomics model that may be used in conjunction with probabilistic disease models to improve or confirm the conclusions of the rules engine.

FIG. 12 shows rules of an illustrative rules engine that combines results from several models, including a fragmentomics model, related to hepatocellular carcinoma (HCC).

FIG. 13 shows rules of an illustrative rules engine that combines models for various abdominal diseases.

### DETAILED DESCRIPTION OF THE INVENTION

A medium-depth whole genome bisulfite sequencing (WGBS) analysis system that integrates classification models trained on disparate datasets will now be described. In the following exemplary description, numerous specific details are set forth in order to provide a more thorough understanding of embodiments of the invention. It will be apparent, however, to an artisan of ordinary skill that the present invention may be practiced without incorporating all aspects of the specific details described herein. In other instances, specific features, quantities, or measurements well known to those of ordinary skill in the art have not been described in detail so as not to obscure the invention. Readers should note that although examples of the invention are set forth herein, the claims, and the full scope of any equivalents, are what define the metes and bounds of the invention.

One or more embodiments of the invention enable a flexible and modular analysis of whole genome bisulfite sequencing (WGBS) data generated from biological samples taken from a patient, such as blood or tissue. The WGBS data may be generated using off-the-shelf technologies and test kits and standard, widely available techniques and data processing pipelines. Bisulfite treatment of DNA identifies methylation patterns, which are highly correlated with many different conditions and diseases.

Embodiments of the invention support single-disease and multi-disease detection workflows within the same modular architecture. For both single-disease detection and multi-disease detection, the platform may utilize a meta-classifier to analyze input data (e.g., clinical metadata and initial feature distributions) to dynamically route specific feature subsets to a corresponding subset of specialized classification models. In a single-indication mode, the meta-classifier may route data to a specialized mixture-of-experts (MoE) ensemble tailored to that indication. For multi-indication detection, the meta-classifier may parallelize this process across multiple distinct MoE ensembles. While the meta-classifier manages the selection and routing of data to the appropriate experts, a separate rules engine may be used for the final adjudication of the individual model outputs to resolve a definitive clinical conclusion. This unified framework allows for maximum scalability and reusability of the sequencing data. It enables broad and accurate diagnostics of many potential diseases and conditions based on a single sequencing of a patient's genome, which may greatly reduce cost compared to custom disease-specific tests and custom sequencing assays.

To support rapid rollout of a wide range of analyses on WGBS data, embodiments of the invention may leverage previous studies and research that have identified methylation markers for various diseases and conditions. Because the data used in this previous work is often not WGBS, the analyses and models that have been generated previously are not directly usable with WGBS data; one of the innovations enabled by embodiments of the invention is to adapt WGBS data to these previous studies, so that previous work can be used and broad diagnostic capabilities can be rapidly deployed that use WGBS data.

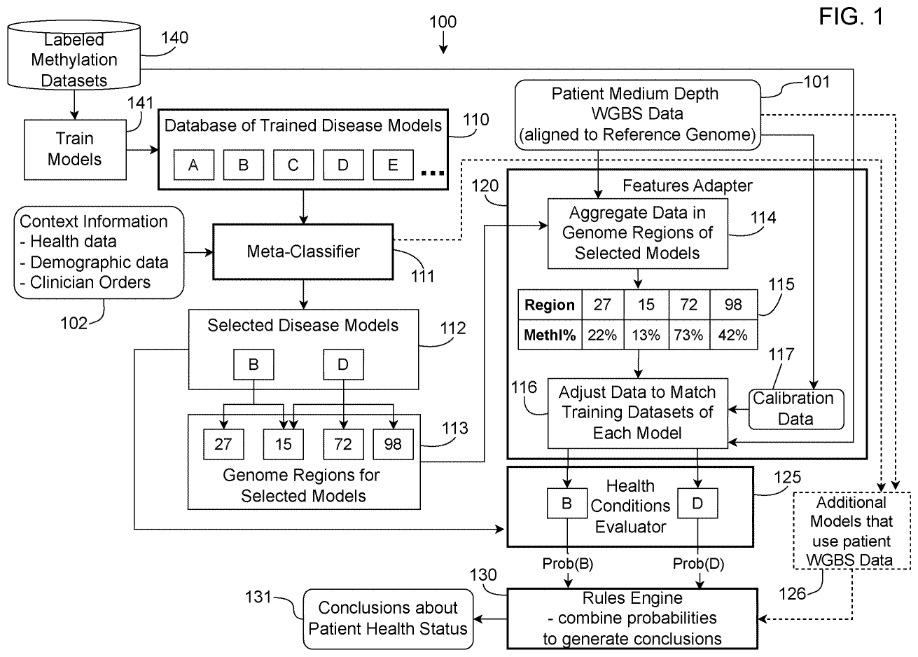

FIG. 1 shows an architectural diagram of components of an illustrative embodiment 100 of the invention. The invention includes several distinct data processing modules that consume, transform, and generate data in an integrated pipeline. These modules may execute on any desired processor or processors, including for example, without limitation, servers, desktop computers, laptop computers, CPUs, GPUs, specialized circuits, or networks of any of these processors. Processing may be performed centrally in one location or distributed over multiple locations.

System 100 receives as input WGBS data 101 for a patient, as well as context information 102 that may provide background on the patient and the clinical objectives for the system's analysis. Through a series of steps described below, the system calculates probabilities that the patient has various conditions or diseases, and it generates a set of overall conclusions 131 about the patient's health status. These conclusions may be transmitted for example to a clinician or facility to guide care decisions or further testing.

WGBS Data 101 may be any genomic sequencing information that includes methylation information. Any sequencing technology or technologies may be used to generate this data. Methylation may be indicated using bisulfite treatment of DNA or using any other chemical transformation that allows a sequencing system to identify areas of methylation.

WGBS data 101 may be medium-depth sequencing data, or in some situations even low-depth data. For example, medium-depth data may be between 10x and 30x. The system's ability to effectively use medium or low depth sequencing data is a key benefit, since it greatly reduces the cost and time for data collection. One of the factors that contributes to the system's ability to use this data is that it aggregates methylation information into regions, as described below, which allows for statistical validity even with relatively low read depth. By transforming stochastic single-site observations at lower depths into deterministic regional features, the system overcomes the signal-to-noise limitations that have historically prevented the commercial implementation of medium-depth whole-genome liquid biopsies.

WGBS data 101 may be obtained by sequencing genetic material from any tissue or tissues of a patient. In one or more embodiments, data 101 may be obtained by sequencing cell-free DNA (cfDNA) that was circulating in a patient's bloodstream or other fluids, for example. cfDNA is already broken into fragments by the body, and these fragments may be sequenced directly (after bisulfite conversion, for example), whereas DNA from other tissues may need to be broken apart as part of the processing pipeline. Moreover, the size and composition of the cfDNA fragments may also provide valuable information that may assist in diagnosis. In one or more embodiments, WGBS data 101 may therefore include data on the individual fragments that were sequenced, as well as aggregate aligned data across the genome.

Context information 102 may be used to guide the processing of the WGBS data 101. For example, it may identify analyses of the WGBS data that are most relevant to the patient given the patient's health status, history, demographics, current treatment, and given any clinician input into the types of tests that may be indicated. Context information 102 may include any information about the patient as well as any orders or other notes from one or more clinicians or other care personnel. This data may be the entirety of or an extract from the patient's medical record or medical records, for example. By processing this context information 102, the system avoids performing unnecessary analyses on data 101, while ensuring that a complete set relevant analyses is performed. Because WGBS data 101 is typically enormous in size (hundreds of gigabytes, for example), intelligently selecting analyses provides a huge gain in efficiency, reducing the cost and time for the system to generate relevant conclusions 131.

System 100 uses a database 110 of trained models, and it selects and executes these models based on patient data inputs. The models are trained on corresponding datasets 140. While these datasets contain methylation information, they are not necessarily WGBS data; instead, many previous studies correlating methylation with diseases or other health conditions have used targeted assays that measure methylation in specific areas of the genome using customized biochemistry panels. This data is not directly comparable to WGBS data since the measurement technology is different. One of the innovative features of system 100 is that it adapts WGBS data to these previous datasets so that models trained on disparate non-WGBS data may be used with patient WGBS information. Datasets 140 may also contain WGBS data, and some models may be trained on a mixture of WGBS and non-WGBS data.

The models in database 110 are typically classifiers that classify a patient as either having or not having a particular health condition associated with each model. The database may contain many different models that address different health conditions, or different aspects of one or more health conditions. One or more embodiments of the invention may use models that are probabilistic classifiers that output a probability that a patient has the condition associated with the model. Some models may be strictly binary in that they decide that a patient either has or does not have the associated condition. (Strictly binary models may be considered a special case of probabilistic classification where the output probability is either 0 or 1.) One or more embodiments may also include one or more models that classify a patient into several categories (potentially more than two), either probabilistically or by selecting a specific category for each patient. The methylation datasets 140 are generally labelled with a binary value that indicates whether each sample patient in the dataset has or does not have the associated health condition. The models 110 are therefore typically trained using supervised learning, although one or more embodiments may also use unsupervised learning techniques instead of or in addition to supervised learning.

Each model in database 110 has an associated health condition, a set of input features, an output that is the probability that a patient has the associated health condition, and a training dataset on which the model is trained. Input features typically include features that indicate the degree of methylation of selected portions of a genome; in one or more embodiments some or all of the models may also have other input features, such as patient characteristics or other features derived from genomic data. As described below, for some models, the system may define expanded genome regions for some or all of the input features for use with WGBS data to provide statistical validity with medium depth sequencing. The training dataset for a model may contain input features and output labels (indicating whether a patient has the health condition or does not) for a set of sample patients. In some situations the training dataset for a model may include data from studies of other diseases or conditions, where patients with those other diseases or conditions are labelled as “negative” (not having the condition of interest for the model); this mixture of data may improve model specificity since it allows the model to differentiate between the condition of interest and potentially confounding or related but distinct conditions.

System 100 may first process context information 102 using a “meta-classifier” 111, which may select which model or models in database 110 should be applied to patient WGBS data 101 based on the context information 102, which may include for example patient etiology, clinical metadata, or specific clinician orders. This meta-classifier 111 may determine which health conditions are most likely or most relevant for the patient, given the context information 102, and it may select models 112 that are related to these conditions. It may also include models for other conditions that may be potentially confounding with the main conditions of interest. Meta-classifier 111 may then identify the union of all of the genome regions 113 associated with the full set of selected models 112 to ensure computational efficiency during subsequent processing. Each model has an associated set of genome regions, where each region corresponds to an input feature of the model. The meta-classifier takes the union of these regions for all selected models. Some regions may appear in more than one selected model (for example, in FIG. 1, models B and D both use region 15), and the meta-classifier may take this into account and not double count a region; this optimization may improve the efficiency of the subsequent processing steps.

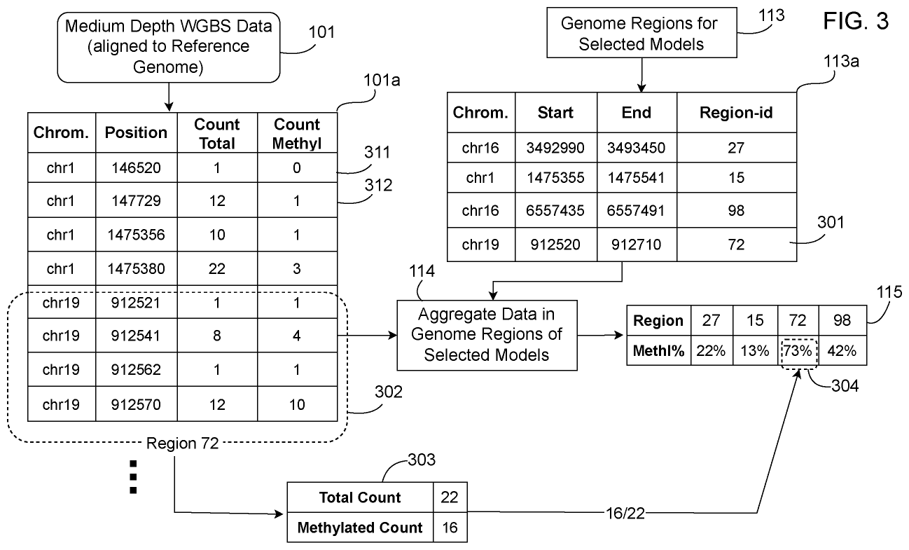

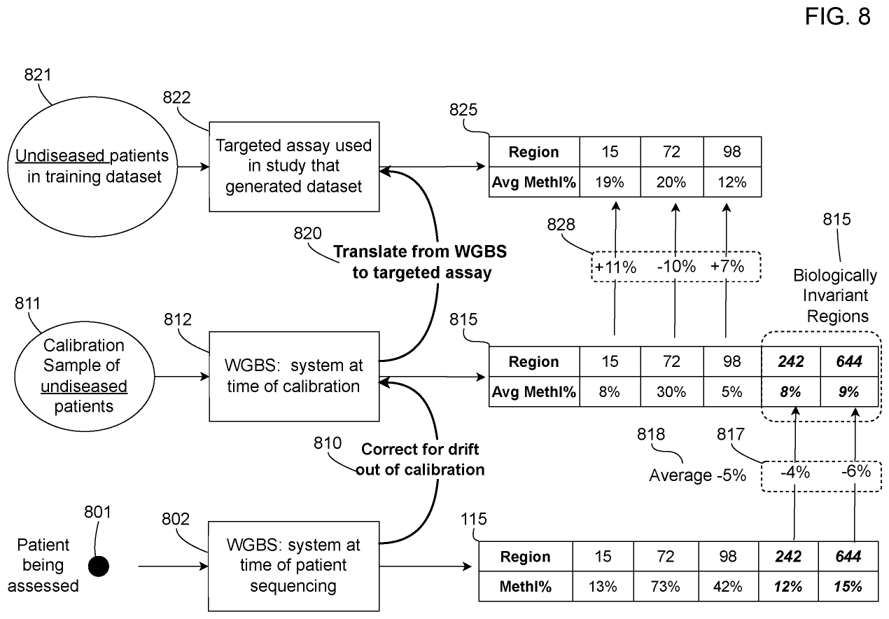

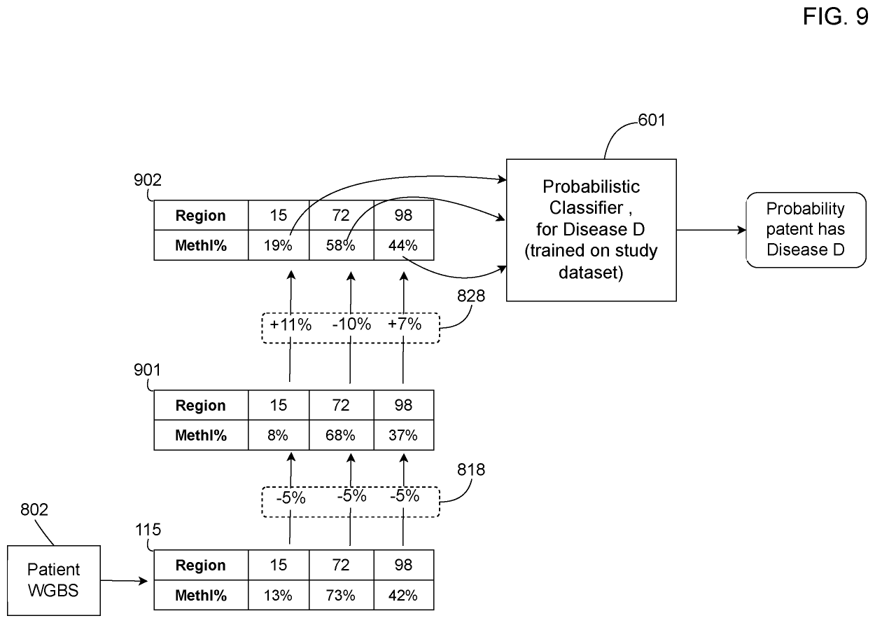

After selecting models 112 and identifying all of the relevant genome regions 113, system 100 may use a features adapter 120 to generate the feature values for the models from the patient WGBS data 101. Features may be generated in two steps: in first step 114, the WGBS data 101 is aggregated into region-level methylation percentages 115 for the regions 113 identified by the meta-classifier; this step is illustrated in FIG. 3. Second, in step 116, the region methylation percentages 115 may be adjusted to adapt the WGBS data 101 to the norms of the training datasets 140 associated with the selected models. This adjustment step 116 may use calibration data 117 from the WGBS system that generated data 101, as described below with respect to FIG. 8 and FIG. 9. The adjusted feature values may then be input into health conditions evaluator 125, which executes selected models 112 to generate probabilities that the patient has each of the health conditions associated with the selected models.

In some embodiments or scenarios, the generated probabilities associated with each selected model may be used directly. In one or more embodiments, the system may perform an additional step of inputting the probabilities into a rules engine 130 that analyzes the full set of probabilities to generate a set of clinical conclusions 131. The rules engine may be particularly valuable when multiple models are selected to evaluate different variations, aspects, or hypotheses, so that the results of these models can be combined into an integrated conclusion.

In one or more embodiments of the invention, one or more additional models 126 may be selected by meta-classifier 111, in addition to the probabilistic classification models described above. These additional models 126 may for example process other aspects of patient WGBS data 102 (and related information) beyond methylation percentages within genomic regions. In some situations, this data may be obtained during the same sequencing process that generates methylation data. For example, as discussed below for some conditions “fragmentomics” features that characterize individual fragments of cfDNA may provide useful complementary information to the probabilities generated by individual condition classification models. Meta-classifier 111 may determine which additional models, if any, are appropriate based on the context information 102. When these additional models are used, output from these models may also be provided to rules engine 130 along with classifier probabilities, and the rules engine's clinical conclusions may be based on the combination of all of this information. Additional model or models 126 may analyze any aspects of patient data 102 and may calculate any desired features or scores from this data.

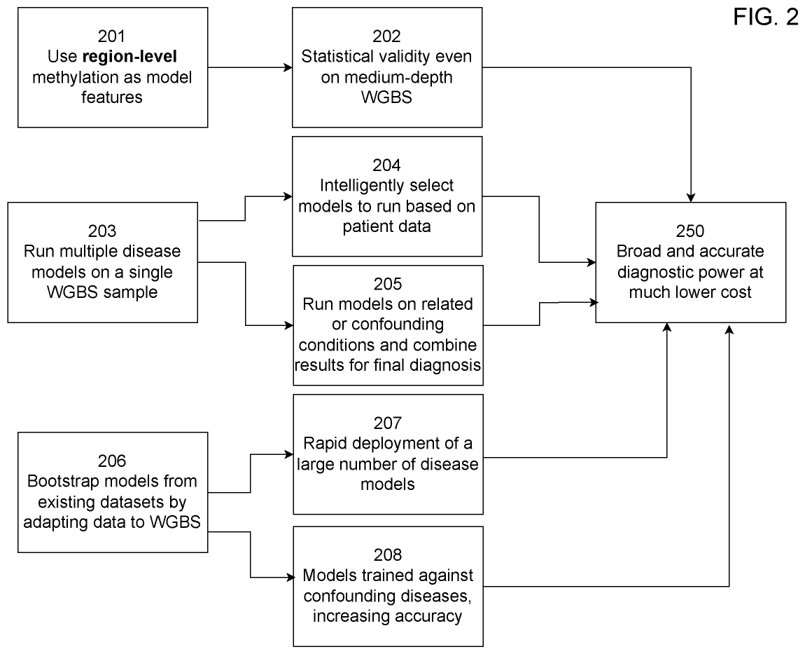

FIG. 2 shows selected aspects of embodiments of the invention that contribute to superior performance, and in particular to benefit 250 of providing broad and accurate diagnostic power at a lower cost. One major contributor 203 to breadth at low cost is the ability to run multiple models for various conditions against a single WGBS sample from a patient. This ‘sequence once, analyze multiple times’ process provides a software-driven, dynamic discovery window that is inherently far more efficient in cost and time than running several, static targeted panels. Efficiency is also enhanced with capability 204 of intelligently selecting models to run based on context data (via the meta-classifier); this approach ensures that processing power and data access are not wasted on irrelevant models. Accuracy of results is enhanced with capability 205, where the meta-classifier may select a suite of models that include models related to the condition(s) of interest as well as models related to confounding or similarly presenting conditions or differential diagnoses. Cost efficiency is also improved using capability 201, where aggregation of methylation data into regions supports use of medium or low depth sequencing data, which takes less time (and therefore less expense) than high depth sequencing. Efficiency, breadth, and accuracy are also enhanced using capability 206 that “bootstraps” models using existing training datasets that may not be WGBS; this approach supports benefit 207 of rapid deployment of a large number of models for different conditions and allows some models to be refined via approach 208 of training the models against cross-disease negative datasets to differentiate the target condition from confounding biological noise. FIG. 3 through FIG. 13 illustrate details of many of the modules and steps shown in the overview diagram of FIG. 1.

FIG. 3 shows an illustrative extract 101a of WGBS patient data that is input into the system 100 and it illustrates aggregation 114 of this data into region-level methylation percentages. Data 101a is generated by the sequencing system, and it is preprocessed to align the data to a reference genome (using standard genomics information tools). This data identifies specific base addresses (consisting of a chromosome identifier and a base offset within the chromosome) and shows the number of total reads of this base by the sequencing system, and the number of these reads that were methylated (identified via bisulfite treatment prior to sequencing). Methylated sites are typically CpG bases. This is the raw patient data that is input into system 100. Cost efficiency may be improved using capability 201, where aggregation of methylation data into regions transforms stochastic single-site observations into deterministic features, allowing for accurate diagnosis at medium sequencing depths. Meta-classifier 111 generates a set of regions 113 that are used in the selected models. Table 113a shows illustrative region definitions; associated with each region is a chromosome and a starting and ending base index. Module 114 then aggregates data 101a within the selected regions 113a. Generally, the selected regions represent a very small portion of the entire genome; as a result, the aggregation 114 can be performed efficiently by reading only the base addresses in data 101a associated with the regions. By avoiding reading the entire WGBS table, the system may greatly reduce the time and expense of processing the data. For example, in data 101a, base addresses 311 and 312 are not part of any of the regions 113a, so they need never be processed. Aggregation of data within a region may be done by adding the methylated read counts of the bases within the region and dividing by the total read counts of the bases within the region. This is illustrated for region 301, where addresses 302 in data 101a are within the boundaries of this region; the sums 303 of methylated and total counts yield methylation percentage 304 in region methylation data 115.

Region-level methylation data 115 is much more statistically robust than individual base methylation data, since averaging methylation over a region reduces the variance due to random variation in reads. This increase in robustness is particularly important for low and medium depth sequencing, since many individual bases may be read only a few times or not at all.

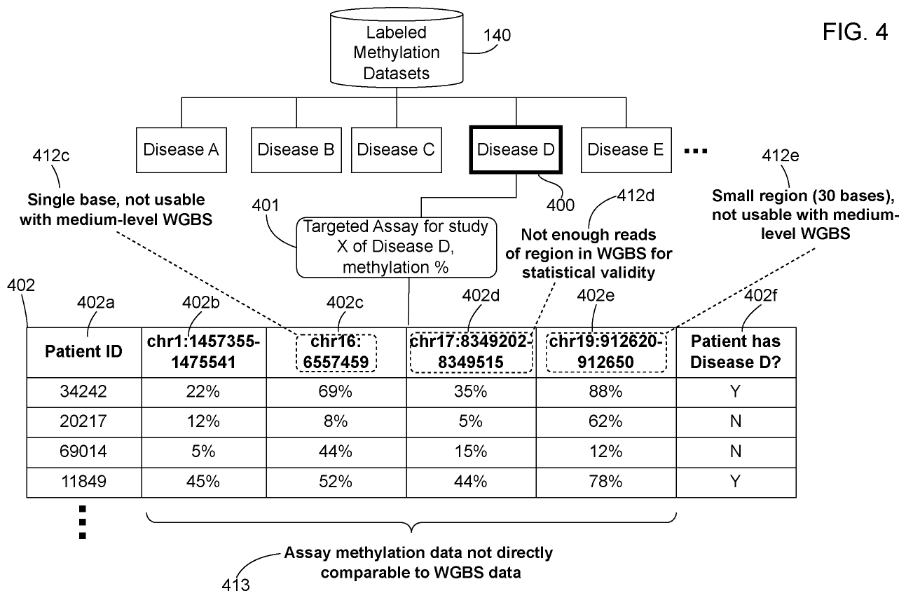

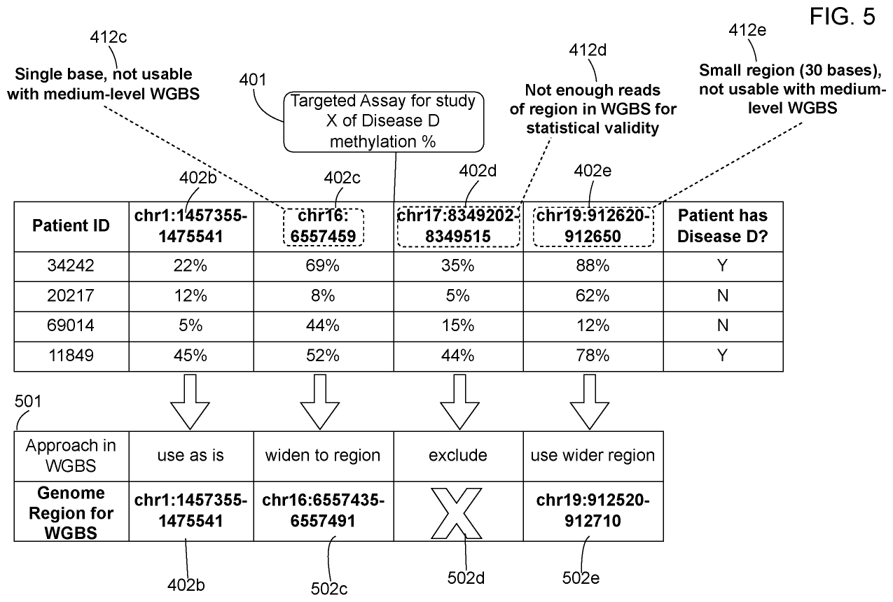

In one or more embodiments, the system may define additional or alternate region-level methylation features; for example, the total number of reads in a region or the number of reads per base in the region may be calculated as well as the average methylation percentage within the region. For regions with a small number of total reads, the system may set the methylation percentage of the region to a “not available” flag to prevent use of a percentage that may be highly unreliable due to a small number of reads. The region methylation features 115 calculated by aggregation 114 are based on the patient's WGBS data. As described above, some or all of the models used by the system may be trained on non-WGBS data. The system must therefore adapt the WGBS data to these models. This process is illustrated in FIG. 4, FIG. 5, FIG. 8, and FIG. 9. FIG. 4 shows a small extract from a sample training dataset for a disease model. This dataset is based on a targeted assay that was used in a study of methylation markers for disease 400; it is not WGBS data. Dataset 401 includes four methylation features 402b, 402c, 402d, and 402e; it also contains a sample patient identifier 402a and a label 402f indicating whether the sample patient has the disease of interest. Each of the four features identifies a portion of a genome and measures the methylation percentage observed for each sample patient in that portion of the genome. Adapting WGBS data to these dataset features needs to address at least two challenges: First, the genomic portions used in the dataset may not be suitable as WGBS regions, as illustrated in issues 412c, 412d, and 412e for columns 402c, 402d, and 402e, respectively. Second, issue 413 is that the assay used to collect the dataset 401 generates data that cannot be directly compared to WGBS data, due to differences in chemistry, lab conditions, sampling methods, equipment, tissue samples used, tissue preparation, bisulfite conversion efficiency, etc. One or more embodiments of the invention address both types of issues to adapt WGBS data to disparate training datasets.

The first type of challenge is that the genomic portions used in the training dataset may be too small or too sparsely sampled to be usable directly as feature regions in WGBS data. Because one or more embodiments of the invention may receive medium depth or low depth WGBS data, regions used to aggregate WGBS methylation data must be sufficiently large to ensure statistical validity. To address the limitations of single-base or narrow legacy features, the system may perform genomic coordinate expansion to wider regions. In some situations, the system may define these regions based on established methylation boundaries, such as Methylation Coherent Blocks (MCB) or regions of proximity-based co-methylation defined in previous studies (e.g., 200 base pairs around a central CpG site). Widening the region ensures that the expanded WGBS-compatible region captures a statistically robust and biologically consistent signal, allowing the system to accurately adapt disparate assay data for use in whole-genome diagnostics This constraint may not be present with the assays used in previous methylation studies; therefore, some of these studies may have used small regions or even individual bases as their methylation features. In illustrative dataset 401, feature 402b is a portion of the genome that is 186 bases wide; this is sufficiently large to be used directly with WGBS data. Feature 402c is a single base (issue 412c), so it cannot be used reliably with WGBS data. Feature 403d is a fairly wide region; however typical WGBS data does not sample with sufficient depth in this region (issue 412d). Feature 403e is a narrow region of only 30 bases (issue 412e), so it cannot be used as-is in WGBS data because it is too small.

FIG. 5 illustrates illustrative approaches 501 to adapting region definitions to WGBS data for the issues 412c, 412d, and 412e of dataset 401. (An approach for dealing with the data comparability issue, issue 413, is illustrated in FIG. 8 and FIG. 9.) Genome region 402b may be used as-is with WGBS data. Feature 402c consists of a single base, which is not usable with medium/low depth WGBS data (issue 412c). An illustrative approach to handle this feature, as well as features with regions that are too narrow, is to define a region that contains the original feature and that is wide enough to be usable with medium/low depth WGBS data. Because methylation values are highly correlated for bases that are near one another, a small genome portion (a single base or a small number of adjacent bases) may be widened and the average methylation percentage in the widened region should at least approximately match that of the original genome portion. (Offsets in values between the widened region and the original genome portion may also be accommodated by the data adjustment process described below with respect to FIG. 8 and FIG. 9.) For feature 402c, the single base is widened to a region 502c for WGBS data. Feature 402e has a similar issue 412e: although it is not a single base, it is a narrow region that is too small to be used effectively with medium/low WGBS data. Using a similar solution, the system defines widened region 502e that contains the original feature area 402e, but has enough bases contained in the widened region that averaging methylation values over this widened region with WGBS data will yield statistically significant results. Finally, for feature 402d, issue 412d identifies that even though the region is sufficiently wide, typical WGBS sequencing does not generate enough reads of this region for accurate results. One approach 502d to this issue is to simply exclude this region from the model for this disease; another approach may be to widen the region even further to the point that it has enough WGBS reads to be usable.

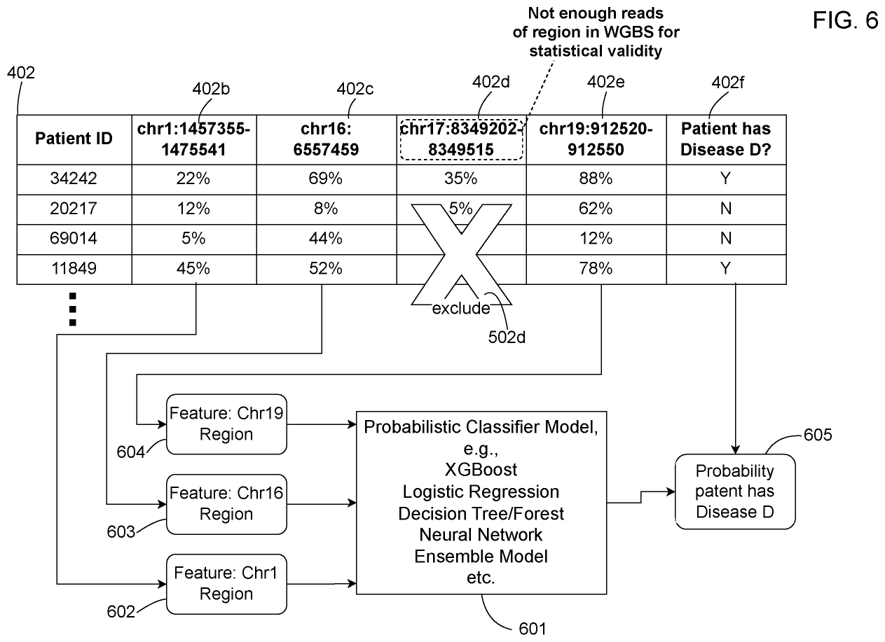

Continuing with the illustrative example of dataset 401 for disease 400, a classification model may be trained on this dataset (with possible extensions as described below). This training process is illustrated in FIG. 6. A machine learning model or an ensemble of models 601 may be defined with input features 602, 603, and 604 corresponding to the features 402b, 402c, and 402e of data 401, (omitting excluded feature or features such as feature 402d), and output 605 that represents the probability that a patient with specified values for these input features has the associated disease. Any type of classification model or models may be used, including for example, without limitation, XGBoost, logistic regression, a decision tree, a decision forest, a neural network, or an ensemble of any combination of these models. In one or more embodiments, multiple models may be trained and the model that performs best on a validation set may be selected for production. Models may be refined and improved over time as additional data is obtained. In particular, as the system is used with patient WGBS data, a dataset of WGBS data for the disease will be accumulated over time, and this data can be added to the original training dataset to improve the classification model. (Combining WGBS data and the original training dataset requires transforming the data to a comparable form, as described below with respect to FIG. 8 and FIG. 9). In some situations, enough WGBS data may be obtained to train one or more models on WGBS data exclusively.

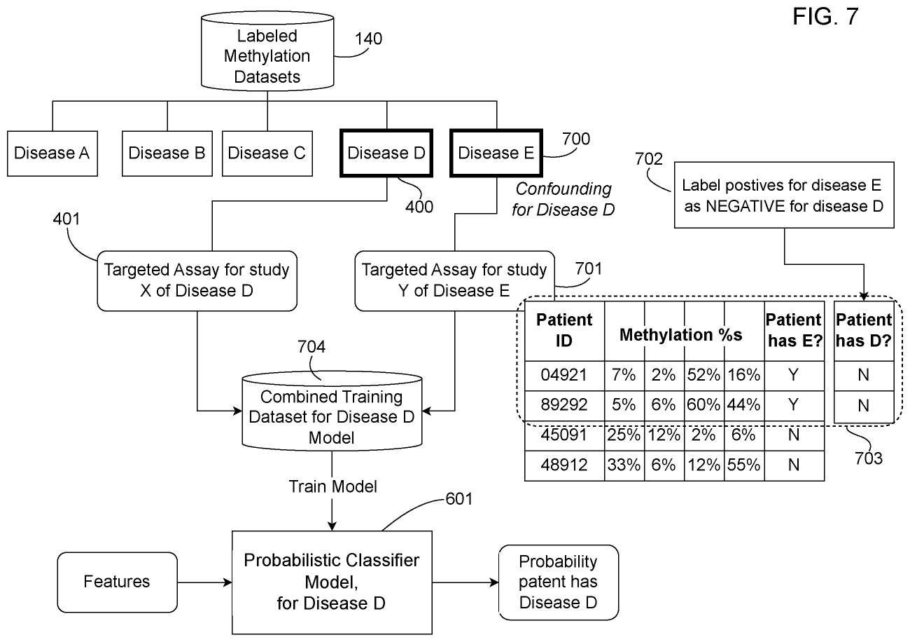

For some health conditions it may be advantageous to train a classification model using data collected for one or more other different conditions. This situation is illustrated in FIG. 7. The objective is to train a classification model for disease 400. It is known that another disease or condition 700 may be confounded with disease 400, for example because it presents with similar symptoms or because test results (including methylation) may be similar. Ideally the classification model 601 for disease 400 should be able to differentiate between disease 400 and disease 700, so that patients with condition 700 are correctly classified as not having disease 400. To achieve this differentiation, in one or more embodiments data for the confounding condition (here, disease 700) may be merged with the dataset for the primary condition of interest (here, disease 400) to form a combined training dataset 704. Dataset 701 for disease 700 may for example have methylation data for a set of sample patients and a label that indicates whether each sample patient has disease 700. (As described previously, this data may not be WGBS in some situations, and may therefore need to be adapted to WGBS sequencing.) The positive samples from dataset 701 (those that have disease 700) may then be labeled in step 702 as being negative for disease 400, to form data 703; this data 703 may then be combined with data 401 to form an expanded training dataset 704 for classification model 601. Expanding the training dataset in this way may improve the specificity of model 601, since it may learn to not misclassify patients with disease 700 as having disease 400. In some situations, there may be multiple confounding conditions and data from each of them may be combined with data 401 to form a training dataset that can differentiate among all of the conditions.

Returning now to issue 413, which is that data collected for training datasets may not be directly comparable to WGBS data, FIG. 8 illustrates an approach that may be used in one or more embodiments to convert WGBS data 115 from a patient 801 to values that can be used with models trained on different disparate datasets. This approach uses a calibration sample 811 corresponding to a group of health patients (that are known to not have any of the health conditions of interest.) This calibration sample is input into the same WGBS system that is used to sequence patient data 115. The methylation data obtained from the calibration sample is aggregated into the same regions used as input features for the model or models of interest, resulting in baseline WGBS region methylation percentages 815. This baseline data 815 can then be compared to the corresponding targeted assay methylation percentages 825 for healthy patients in the model's training sample. This analysis compares healthy patients to healthy patients so that the methylation effects of a disease or health condition do not affect the comparison. Because the differences in measured values between WGBS and a different assay may vary by genome region, the comparison is performed on a region-by-region basis, yielding offsets 828. In translation process 820, these offsets may be added to region methylation percentages from WGBS data to generate data that is comparable to a training dataset assay, and that may therefore be used with a classification model trained on that training dataset.

In one or more embodiments, an additional correction 810 may be made for drift of a WGBS system from its calibrated state. Because sequencing is time-consuming and expensive, calibration samples are not necessarily run through a sequencing system before processing every patient sample. Therefore, the sequencing system (including the hardware and all chemical and environmental factors) may drift away from its state at the time of the last calibration sample. One or more embodiments of the invention may measure this drift by using biologically invariant regions 815 of the genome that have known methylation percentages, regardless of the patient or the health condition of the patient. Because methylation of these regions should be a constant across patients and across time, differences 817 observed in methylation percentages for these invariant regions (between the WGBS system 802 at the time of patient sequencing, and the WGBS system 812 at the time of calibration) are due to the aforementioned drift. If these differences are measured for multiple biologically invariant regions, the average difference 818 may be used as an offset to correct for drift in the regions of interest. In one or more embodiments, this adjustment for drift may be made only if the drift is within a certain valid range of values; if the drift is very small—below this valid range—no adjustment may be made, and conversely if the drift is very large—above this valid range—then the system may mark the patient sequencing data as invalid because the sequencing system has gone too far out of calibration or because the data may be corrupted by noise or measurement errors.

FIG. 9 continues the example of FIG. 8 to show applying the corrections 810 and 820 to patient WGBS data 115. First the average calibration drift factor 818 is added to each region methylation percentage, to generate data 901. Then individual region offsets 828 are added to each region methylation percentage in 901 to generate the final data 902 that is input into classifier 601.

The specific correction steps illustrated in FIGS. 8 and 9 are illustrative; any method that adapts WGBS data to non-WGBS training data (or even to training data on a different WGBS system) is in keeping with the spirit of the invention.

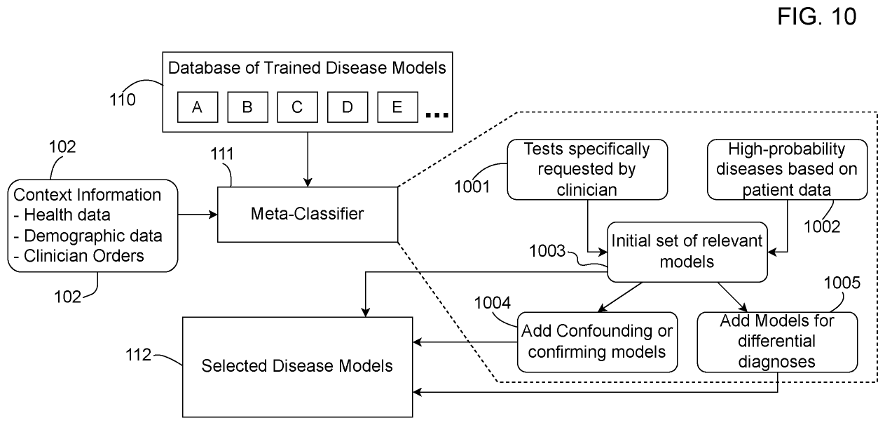

Turning now to the meta-classifier that initially selects a set of models to use to analyze patient WGBS data, FIG. 10 shows an illustrative framework that may be used for meta-classification in one or more embodiments of the invention. The meta-classifier 111 may process context information 102 to first identify two broad categories of models: models corresponding to tests or inquiries 1001 specifically requested or suggested by a clinician, and models corresponding to diseases or conditions 1002 that may be likely given the patient's history, demographics, and known health conditions. For example, patients may have known problems that are correlated with certain additional diseases, or some diseases may be likely given a patient's age, sex, habits, or family history. These two factors 1001 and 1002 may be combined to yield a preliminary set of models 1003. Meta-classifier 111 may then expand this preliminary set 1003 to include models 1004 for conditions that are confounding or confirming for the initial set, and to include models 1005 that may contribute to a differential diagnosis, for example by testing for alternative explanations to patient symptoms. The complete set of models including 1003, 1004, and 1005 may then be used as selected disease models 112.

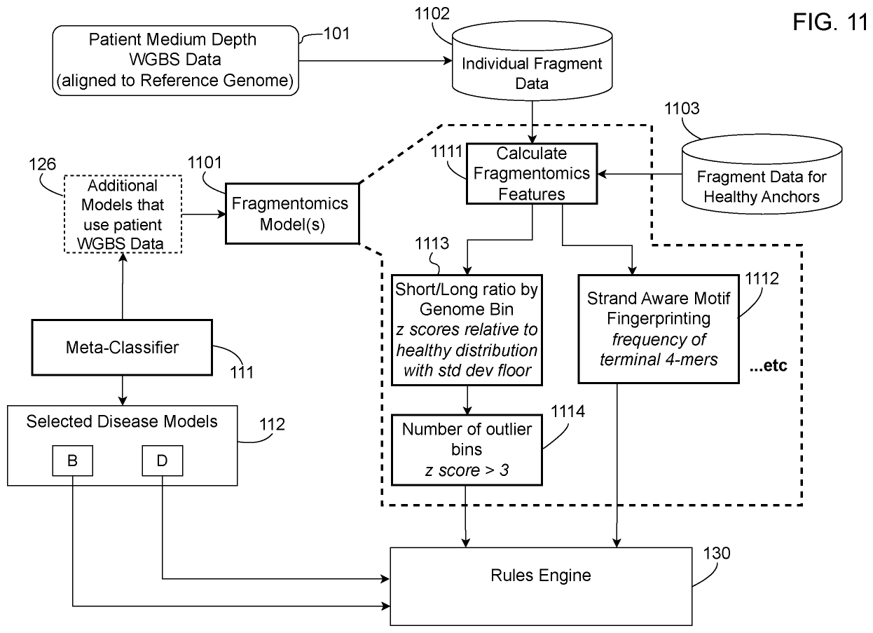

The classification models described above with respect to FIG. 3 through FIG. 10 are trained on methylation percentages for associated genomic regions, and they output probabilities that the patient has the condition associated with each model. In some situations, other aspects of genome sequencing data (or related information) besides methylation levels may also provide useful diagnostic information. For example, it is known that the fragment size of cfDNA may provide an indication of whether a patient has cancer, since cfDNA fragments from cancer tend to be shorter on average. Other fragment features, such as the pattern of nucleotides at the fragment ends, may also be correlated with cancer or other diseases. Therefore, in one or more embodiments, the meta-classifier may run one or more additional models 126 to capture some of these aspects of genomic signatures that are not covered by condition-specific methylation-based probabilistic classifiers. FIG. 11 illustrates an example where meta-classifier 111 adds a fragmentomics model 1101 to the set of models that analyze patient genomic data 101. The fragmentomics model 1101 may calculate any features 1111 that reflect how DNA is fragmented into cfDNA and what the characteristics of these fragments are. Many such measures have been studied in previous research, and one or more embodiments may incorporate any of these known measures, or variants thereof, into one or more fragmentomics models. For example, Chen et. al. developed a score that is correlated with the risk for HCC that is based on four fragmentation features: extent of 5-hydroxymethylation, nucleosome footprint, 4-mer 5′-end motif patterns, and ratio of small fragments (<150 bps) to large fragments (>150 bps) within bins across the full genome. (See “Cell-free DNA testing for early hepatocellular carcinoma surveillance”, Chen, Lei et al., eBioMedicine, Volume 100, 104962). Any or all of these features may be used in fragmentomics model 1101 or in similar models.

FIG. 11 shows two fragmentomics features that may be calculated by illustrative fragmentomics model 1101. These features may be based on individual fragment data 1102 that is included within patient data 101, and on corresponding fragment data 1103 for a set of healthy patients. The features may, for example, show differences in fragment characteristics between the patient being tested and the healthy controls 1103. First, in step 1112 the fragmentomics model 1101 may perform strand-aware end motif fingerprinting, where the 4-mers at both ends of fragments are extracted and normalized to the genomic forward strand to ensure biological consistency across sequencing reads. The result of this process may be for example a 512-dimensional vector with the 4-mer frequency distributions across all fragments 1102 for the 5′ end (256 possible 4-mers) and for the 3′ end (another 256 possible 4-mers). To measure fragment size distribution, in step 1113 the fragmentomics model 1101 may first divide the entire genome into bins, and within each bin calculate the ratio of small fragments (150 bp or smaller) to large fragments (greater than 150 bp) for each patient of the healthy controls. The same calculation may be performed for the patient being tested. The patient's ratios may then be normalized to the healthy controls by converting each bin's ratio into a z-score by subtracting the mean ratio within the bin for the healthy controls and dividing by the standard deviation within the bin. A high z-score for a bin represents an unusually large ratio of small fragments, which can be an indicator of malignancy.

To improve statistical stability, in one or more embodiments step 1113 may set a floor on the standard deviation used to normalize patient bin ratios to z-scores. This assists stability because in the Healthy Anchors dataset 1103, some genomic bins may have naturally low variance (for example, because the dataset 1103 contains relatively few patients), which causes the Z-score to “explode” mathematically. The standard deviation floor stabilizes the denominator to ensure the results remain statistically reliable for individual diagnostics.

Illustrative bins that may be used for the short-to-long ratio z-score calculations are fixed 1 MB and 5 MB windows that tile the entire genome. (The genome may be divided into bins at both resolutions.) This allows the system to map “islands” of fragmentomic shifts to specific chromosomal locations rather than just looking at a single average for the whole patient.

Calculations 1113 may generate two vectors of z-scores, one for 1 MB bins and one for 5 MB bins. These vectors may be further simplified into a fragment size score 1114, for example by counting the number of bins with z-scores exceeding a threshold value, such as z>3.0 for example. This high z-score count may be input into rules engine 130, along with features derived from 4-mer fingerprinting 1112 and from any other calculations made on fragment data.

The feature calculations 1112, 1113, and 1114 are illustrative; any features derived from genomic data, including individual fragment information 1102, may be used in one or more embodiments of the invention. Other illustrative fragmentomics features may include for example, without limitation: Motif Diversity Scores that quantifying the entropy or “shuffling” of the 4-mer terminal motifs across the entire genome, Nucleosome Footprinting that measures the protection patterns of DNA fragments to infer transcription factor binding and gene expression states, and End-Distance Metrics that analyze the precise distance of fragment ends from known nucleosome centers.

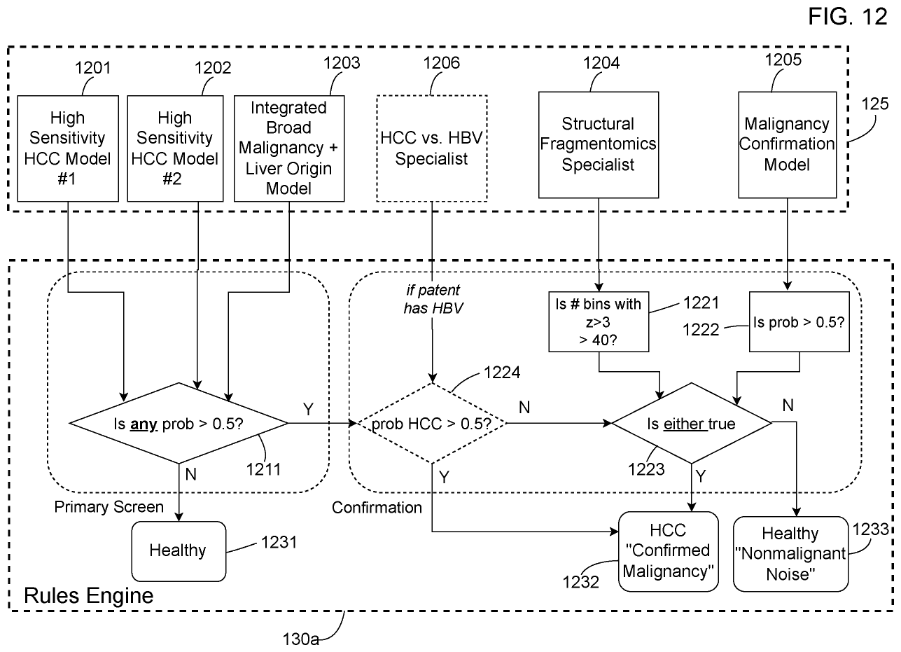

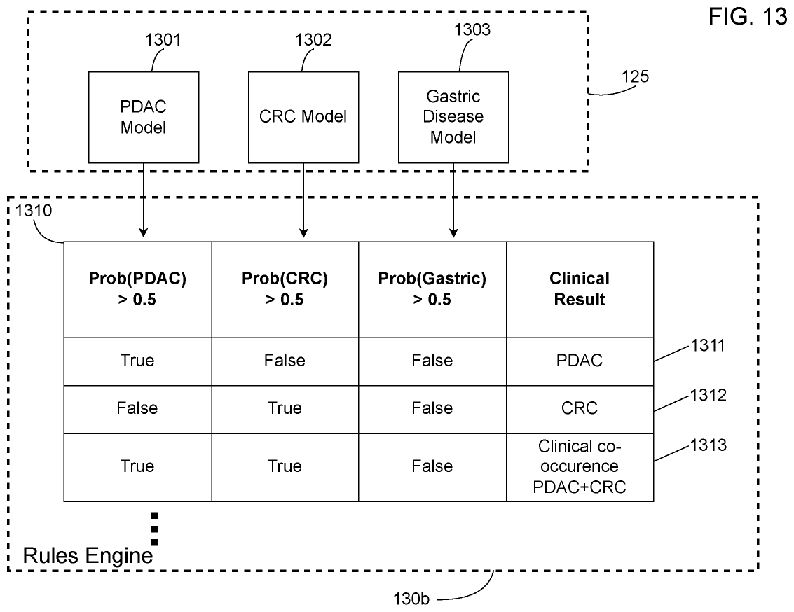

Once disease models 112 are selected by the meta-classifier, these models are run against a patient's WGBS data (which may be transformed to be compatible with model inputs), to generate a probability for each condition associated with each model. The meta-classifier may also identify one or more other models, such as a fragmentomics model, that may generate additional features to improve or confirm a diagnosis. Although the probabilities and features themselves may be of interest, and in some situations may be sufficient, particularly for scenarios with large numbers of models it may be helpful to process the probabilities and features using rules engine 130 to generate an integrated set of conclusions about the patient's condition. The rules engine may apply any algorithm or set of rules to transform probabilities and features into conclusions; in one or more embodiments the rules engine itself may be a machine learning model or may use machine learning or other AI techniques to generate conclusions. In other scenarios rules may be specifically coded to handle the conditions of interest. FIG. 12 and FIG. 13 show two illustrative rules engines: rules engine 130a in FIG. 12 for hepatocellular carcinoma (HCC), and rules engine 130b in FIG. 13 for a gastrointestinal panel.

In FIG. 12, six models 1201 through 1206 may be selected to evaluate a patient's WGBS for HCC and related conditions. Models 1201 and 1202 are “high-sensitivity HCC specialist” (Negative Predictive Value (NPV) anchor) models. These models act as a “wide net” (Negative Predictive Value gating) since they are trained to detect the earliest or most subtle epigenetic shifts. Model 1203 performs an integrated analysis. It evaluates both liver-specific epigenetic signals (organ-of-origin) and broad malignancy-associated signals. It provides the “consensus context.” In a first primary screening stage, rules engine 130a determines whether any of the three models 1201, 1202, and 1203 identifies a potential malignancy with an associated probability of greater than 0.5. If this test 1211 is negative (none of the three primary screening models suggest malignancy, the patient status 1231 is classified as “Healthy”. If any of the primary screening models are positive, rules engine 130a proceeds to a confirmation stage that uses models 1204 and 1205, and potentially model 1206.

Model 1206 is applied only if it is known (for example from context information 102) that the patient has pre-existing Hepatitis-B (HBV). This model 1206 is an HBV specialist that is not confounded by HCC when HBV is present. If the patient has HBV, and if any of the models in the preliminary screen stage are positive, then the HCC probability from model 1206 is tested in test 1224. If this test is positive for HCC, then the rules engine concludes that the patient status 1232 is confirmed HCC malignancy. If test 1224 is negative, then the rules engine invokes the other models 1204 and 1205 to confirm (or refute) malignancy. These additional confirmation tests are necessary because model 1206 may miss early cancers; it acts as a high-specificity “rule-in” gate, but it cannot definitively rule out malignancy.

Model 1205 is a “malignancy auditor” that acts as a high-specificity (Positive Predictive Value (PPV) anchor) “forensic filter” to verify if the generalist signals are actually derived from a tumor. Model 1204 is a Structural Fragmentomics Specialist that provides a complementary high-specificity (PPV) filter by analyzing fragment-length anomalies and genomic positioning, providing a multi-modal verification that distinguishes true malignancy from the structural “noise” of liver disease scarring. In the confirmation stage, after a test by model 1206 if applicable, model 1204 confirms malignancy for example if the number of genomic bins with a z score above 3.0 for the ratio of short to long fragments exceeds 40, since an abnormally high fraction of short fragments is indicative of malignancy. Model 1205 confirms malignancy if it reports a probability of HCC of greater than 0.5. If either model 1204 or model 1205 confirms malignancy, then test 1223 concludes that the patient status 1232 is a confirmed HCC malignancy; otherwise the patient is classified with status 1233 Healthy, with a presumed false positive from the primary screen due to noise.

In this example, the system combines high-sensitivity models 1201 and 1202 and a high-specificity model 1203 to achieve higher classification accuracy. The other models 1204, 1205, and 1206 may be used to handle ambiguous cases. This illustrates that while individual specialist models, each derived from established published biomarkers, miss different samples, the rules engine adjudication of the combined set of probabilities and features resolves these conflicts deterministically.

FIG. 13 illustrates three models selected for a general gastrointestinal panel: model 1301 tests for pancreatic ductal adenocarcinoma (PDAC), model 1302 tests for colorectal cancer (CRC), and model 1303 tests for non-cancerous gastric disease. Rules engine 130b consolidates binary verdicts from these independent, disease-specific specialist models. A simple union of the verdicts are returned as the final clinical finding, as illustrated in a portion of table 1310. For example, if only model 1301 shows a probability greater than 0.5, rule 1311 concludes that the patient has PDAC. If only model 1302 shows a probability greater than 0.5, rule 1312 concludes that the patient has CDC. If model 1301 and 1302 both show probabilities above 0.5, rule 1313 concludes that the patient has both CDC and PDAC. In this scenario the system provides a comprehensive health audit where specialized MoE ensembles for different indications operate independently. Therefore, if multiple ensembles return a positive result, as in rule 1313, the rules engine may report both findings simultaneously. As another example, a patient may biologically have two conditions, such as underlying cirrhosis and HCC, that may share certain biomarkers. The system may use specialized expert models to resolve distinct signals for these two conditions and report that the patient has both conditions, rather than being confused by overlapping signals.

To validate the efficacy of the HCC Mixture of Experts (MoE) embodiment (illustrated for example in FIG. 12), the system was subjected to multi-stage pre-clinical validation using both tissue-derived and cell-free DNA (cfDNA) datasets.

Initial feasibility was established using a tissue-derived dataset consisting of 24 samples (12 matched pairs of HCC tumor and adjacent non-tumor tissue). The MoE framework successfully differentiated malignant signatures from matched normal backgrounds across the cohort. Further validation was conducted using an independent immune-response cfDNA dataset consisting entirely of non-malignant samples; the MoE ensemble correctly classified all samples as “negative,” demonstrating high specificity and a lack of confounding by systemic immune-derived epigenetic signals.

Finally, the system was validated against a retrospective plasma cfDNA cohort of biobank samples, including biopsy-confirmed HCC cases and clinical controls representing various stages of chronic liver disease (CLD), cirrhosis, and viral hepatitis (HBV/HCV). By integrating the MCB Specialist (NPV anchor) with orthogonal Fragmentomics verification (Model 1204), the MoE architecture achieved a performance benchmark of >90% sensitivity and >90% specificity. These results demonstrate that the hierarchical adjudication effectively resolves the non-linear complexity of liver disease scarring and viral-induced drift, which typically generate false positives in traditional linear biostatistical models.

In one or more embodiments of the invention, patient WGBS data, patient context information, and the diagnoses generated by the system for each patient may be logged into an archive database. In some cases, additional information may be added to this archive after the system's initial diagnoses, for example with health status updates or outcomes for patients that the system has processed. Over time, this archive database may grow in size and scope as data is collected for a large number of patients, and the archive database itself may become a valuable source of training data for the development of new models and for the discovery of new biomarkers for diseases. For example, initial deployment of a health condition model for a condition such as HCC may rely on previous studies that have identified methylation regions of interest, using targeted assays instead of WGBS; however, when the system has accumulated a large number of patients and has archived these patients' full sequencing data, it may be possible to train a more comprehensive model for HCC that looks at the entire genome. Moreover, entirely new additional models may be discovered by correlating other patient information, such as future health conditions after initial WGBS processing, with data in patient genomes. Some of these additional models may use new features extracted from WBGS data that represent previously unknown biomarkers for health conditions associated with the additional models. The system therefore supports continuous learning, and it becomes increasingly valuable as it is used over time.

While the invention herein disclosed has been described by means of specific embodiments and applications thereof, numerous modifications and variations could be made thereto by those skilled in the art without departing from the scope of the invention set forth in the claims.

What is claimed is:

1. A medium-depth whole genome bisulfite sequencing analysis system that integrates classification models trained on disparate datasets, comprising:
a multiplicity of classification models, wherein:
each classification model of the multiplicity of classification models comprises:
a health condition;
input features, wherein each input feature of the input features comprises:
a methylation percentage associated with a portion of a genome; and
a genome region containing the portion of the genome, wherein the genome region is used with whole genome sequencing data;
an output comprising a probability that a patient has the health condition;
training samples, wherein each training sample of the training samples comprises:
a sample patient;
the input features associated with the sample patient; and
a label indicating whether the sample patient has the health condition;
a machine learning model trained on the training samples to calculate the output from the input features;
a meta-classifier configured to:
receive context information associated with a patient, comprising one or more of:
health data associated with the patient;
demographic data associated with the patient;
clinician orders associated with the patient;
select one or more selected models from the multiplicity of classification models based on the context information; and
identify selected regions of a genome as a union of genome regions associated with all input features associated with the one or more selected models;
a features adapter configured to:
receive methylation data generated by whole genome bisulfite sequencing of one or more biological samples from the patient;
aggregate the methylation data in one or more selected regions of the selected regions to calculate a methylation percentage for each selected region of the one or more selected regions;
for each selected model of the one or more selected models:
translate the methylation percentage for each selected region corresponding to the each selected model into a value that is compatible with the training samples associated with the each selected model, to yield patient feature values for the input features associated with the each selected model;
a health conditions engine configured to:
input the patient feature values into the one or more selected models to calculate probabilities that the patient has health conditions associated with the one or more selected models.

2. The medium-depth whole genome bisulfite sequencing analysis system that integrates classification models trained on disparate datasets of claim 1, further comprising:
a rules engine configured to:
apply one or more rules to the probabilities that patient has the health conditions, to generate conclusions about health status of the patient.

3. The medium-depth whole genome bisulfite sequencing analysis system that integrates classification models trained on disparate datasets of claim 2, wherein:
said meta-classifier selects a plurality of selected models from the multiplicity of classification models; and,
the rules engine is further configured to generate a union of binary verdicts from said plurality of selected models as said conclusions about health status of the patient.

4. The medium-depth whole genome bisulfite sequencing analysis system that integrates classification models trained on disparate datasets of claim 1, wherein:
the machine learning model comprises one or more of:
XGBoost;
a decision tree;
a random forest;
logistic regression;
a neural network.

5. The medium-depth whole genome bisulfite sequencing analysis system that integrates classification models trained on disparate datasets of claim 1, wherein:
the whole genome bisulfite sequencing comprises medium-depth sequencing.

6. The medium-depth whole genome bisulfite sequencing analysis system that integrates classification models trained on disparate datasets of claim 5, wherein:
the medium-depth sequencing comprises sequencing depth between 10x and 30x, inclusive.

7. The medium-depth whole genome bisulfite sequencing analysis system that integrates classification models trained on disparate datasets of claim 1, wherein:
when the portion of the genome associated with an input feature of a classification model comprises a single base address, the genome region containing the portion of the genome comprises a contiguous region of a plurality of base addresses that contains the single base address.

8. The medium-depth whole genome bisulfite sequencing analysis system that integrates classification models trained on disparate datasets of claim 1, wherein translate the methylation percentage for each selected region corresponding to the each selected model into a value that is compatible with the training samples associated with the each selected model comprises:
adjust the methylation percentage for a difference between the whole genome bisulfite sequencing of tissue samples from the patient and whole genome bisulfite sequencing of a set of calibration samples; and
further adjust the methylation percentage for a difference between the whole genome bisulfite sequencing of the set of calibration samples and the training samples.

9. The medium-depth whole genome bisulfite sequencing analysis system that integrates classification models trained on disparate datasets of claim 8, wherein adjust the methylation percentage for a difference between the whole genome bisulfite sequencing of tissue samples from the patient and whole genome bisulfite sequencing of a set of calibration samples comprises:
calculate a patient average methylation percentage in one or more biologically invariant regions from the whole genome bisulfite sequencing of tissue samples from the patient;
calculate a calibration average methylation percentage in the one or more biologically invariant regions from the whole genome bisulfite sequencing of the set of calibration samples; and,
add a difference between the calibration average methylation percentage and the patient average methylation percentage to the methylation percentage for each selected region.

10. The medium-depth whole genome bisulfite sequencing analysis system that integrates classification models trained on disparate datasets of claim 9, wherein adjust the methylation percentage for a difference between the whole genome bisulfite sequencing of tissue samples from the patient and whole genome bisulfite sequencing of a set of calibration samples further comprises:
when said difference between the calibration average methylation percentage and the patient average methylation percentage is greater than a valid range of values, mark the whole genome bisulfite sequencing of one or more biological samples from the patient as invalid.

11. The medium-depth whole genome bisulfite sequencing analysis system that integrates classification models trained on disparate datasets of claim 8, wherein further adjust the methylation percentage for a difference between the whole genome bisulfite sequencing of the set of calibration samples and the training samples comprises:
calculate a calibration average methylation percentage in the methylation percentage for each selected region from the whole genome bisulfite sequencing of the set of calibration samples;
calculate a training set average methylation percentage for each selected region from the training samples wherein the sample patient does not have the health condition; and,
add a difference between the training set average methylation percentage and the calibration average methylation percentage to the methylation percentage for each selected region.

12. The medium-depth whole genome bisulfite sequencing analysis system that integrates classification models trained on disparate datasets of claim 1, wherein:
the training samples associated with at least one classification model of the multiplicity of classification models comprises additional training samples from a different classification model of the multiplicity of classification models, wherein the sample patient associated with the additional training samples has the health condition associated with the different classification model and is labelled as not having the health condition associated with the at least one classification model.

13. The medium-depth whole genome bisulfite sequencing analysis system that integrates classification models trained on disparate datasets of claim 1, wherein the features adapter extracts and processes methylation data only in said union of genome regions associated with all input features associated with the one or more selected models.

14. The medium-depth whole genome bisulfite sequencing analysis system that integrates classification models trained on disparate datasets of claim 13, wherein the features adapter performs a single-pass selective extraction of said methylation data only in said union of genome regions.

15. The medium-depth whole genome bisulfite sequencing analysis system that integrates classification models trained on disparate datasets of claim 2, wherein:
the meta-classifier is further configured to select a fragmentomics model;
the fragmentomics model is configured to calculate one or more fragmentomics feature values from said whole genome bisulfite sequencing of one or more biological samples from the patient; and,
the rules engine is further configured to:
apply said one or more rules to the probabilities that patient has the health conditions and to said one or more fragmentomics feature values.

16. The medium-depth whole genome bisulfite sequencing analysis system that integrates classification models trained on disparate datasets of claim 15, wherein said one or more fragmentomics feature values comprise:
a measure of a ratio of short fragments to long fragments in said whole genome bisulfite sequencing of one or more biological samples from the patient.

17. The medium-depth whole genome bisulfite sequencing analysis system that integrates classification models trained on disparate datasets of claim 16, wherein:
said fragmentomics model divides a human genome into one or more series of genome bins; and,
said measure of the ratio of short fragments to long fragments comprises a number of said one or more series of genome bins wherein a normalized z-score of a ratio of short fragments to long fragments within a genome bin exceeds a threshold value.

18. The medium-depth whole genome bisulfite sequencing analysis system that integrates classification models trained on disparate datasets of claim 17, wherein said normalized z-score is calculated using a mean value and a standard deviation value within said genome bin calculated using genome data from a set of healthy patients.

19. The medium-depth whole genome bisulfite sequencing analysis system that integrates classification models trained on disparate datasets of claim 18, wherein said normalized z-score is further calculated using a floor value for said standard deviation value within said genome bin.

20. The medium-depth whole genome bisulfite sequencing analysis system that integrates classification models trained on disparate datasets of claim 1, further comprising:
an archive database comprising said whole genome bisulfite sequencing of one or more biological samples from a multiplicity of patients; and,
one or more additional models trained on said archive database;
wherein:
one or more of said one or more additional models uses one or more new features extracted from whole genome bisulfite sequencing data that represent previously unknown biomarkers for health conditions associated with the one or more of said one or more additional models.
